(19) United States
(12) Patent Application Publication
(10) Pub. No.: US 2014/0243626 A1
(43) Pub. Date: Aug. 28, 2014

(54) POWER REDUCTION FOR OXIMETRY SENSOR OPERATION

(71) Applicant: COVIDIEN LP, Mansfield, MA (US)

(72) Inventors: Kalpathy Krishnan, Boulder, CO (US); Thomas C. Geske, Erie, CO (US)

(73) Assignee: Covidien LP, Mansfield, MA (US)

(21) Appl. No.: 13/781,227

(22) Filed: Feb. 28, 2013

(51) Int. Cl. A61B 5/1455

(52) U.S. Cl. CPC A61B 5/14552
USPC 600/323

(57) ABSTRACT

Systems, methods, and devices are provided for reducing power consumption of a medical sensor system. In an embodiment, a patient monitor may include driving circuitry to drive an emitter of a sensor to emit light into a patient in accordance with a power-reducing timing cycle. For example, the power-reducing timing cycle may include emitting periods in which the emitter emits light and dark periods in which the emitter does not emit light. In certain embodiments, the dark periods may occur for a longer duration than the emitting periods.

### BACKGROUND

The present disclosure relates generally to medical monitoring systems and, more particularly, to non-invasive medical monitoring systems employing optical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of devices have been developed for non-invasively monitoring physiological characteristics of patients. For example, a pulse oximetry system may detect various patient blood flow characteristics, such as the blood-oxygen saturation of hemoglobin in arterial blood, the volume of individual blood pulsations supplying the tissue, and/or the rate of blood pulsations corresponding to each heart beat of a patient. To determine these physiological characteristics, light may be emitted into patient tissue, where the light may be scattered and/or absorbed in a manner dependent on such physiological characteristics.

Non-invasive medical sensor systems may include a medical sensor and a patient monitor. The patient monitor may send driving signals to an emitter in the sensor, causing the sensor to emit light into pulsatile patient tissue. A detector in the medical sensor may detect the light after it has passed through the patient tissue, generating an electrical current proportional to the amount of detected light. This electrical current, referred to as a photocurrent, may be received by the patient monitor and converted into a voltage signal using a current-to-voltage (I-V) converter. The resulting voltage signal may be analyzed to determine certain physiological characteristics of the patient tissue. The voltage signal, however, often contains noise components, and the patient monitor may not be able to accurately determine the physiological characteristics from a voltage signal with low signal-to-noise ratio (SNR).

To improve the SNR of the voltage signal, a medical sensor system, such as a pulse oximeter system, will typically drive the emitter with a large amount of current. The large drive current may cause the emitter to generate more light, which may improve the SNR. Unfortunately, increasing the SNR in this manner may cause the medical sensor system to consume an undesirably large amount of power. Accordingly, it may be desirable to provide medical sensor systems that consume less power without negatively compromising the SNR of the voltage signal.

### BRIEF DESCRIPTION OF THE DRAWINGS

Advantages of the disclosed techniques may become apparent upon reading the following detailed description and upon reference to the drawings in which:

FIG. 1 is a perspective view of a non-invasive medical sensor system including a patient monitor and a medical sensor, in accordance with an embodiment;

FIG. 2 is a block diagram of the medical sensor system of FIG. 1, in accordance with an embodiment;

FIG. 3 is a process flow diagram of a method of operation that may be employed by the medical sensor system of FIG. 1 to reduce the power consumption of the medical sensor system of FIG. 1, in accordance with an embodiment;

FIG. 4 is a timing diagram representing emitter excitation that may be employed by the medical sensor system of FIG. 1 to digitize measurements of a signal obtained by the sensor of the medical sensor system of FIG. 1, in accordance with an embodiment;

FIG. 5 is a process flow diagram of a method of operation that may be employed by the sensor system of FIG. 1 to measure one or more operating parameters of the sensor of the medical sensor system of FIG. 1, in accordance with an embodiment; and

FIG. 6 is a timing diagram representing emitter excitation that may be employed by the sensor system of FIG. 1 to measure one or more operating parameters of the sensor of the medical sensor system of FIG. 1, in accordance with an embodiment;

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, a medical sensor system may non-invasively monitor physiological characteristics of a patient. In particular, a medical sensor system may include a patient monitor that may transmit driving signals to a sensor to cause an emitter of the sensor to emit light into a patient. Additionally, as described above, the patient monitor may drive the emitter at a current level proportional to a desired SNR. That is, a higher current level may be selected to increase the SNR. However, this may also increase the power need of the medical sensor system. Moreover, a patient monitor typically analyzes signals received from the sensor to determine whether the SNR is sufficient and whether the current level should be adjusted. Unfortunately, processing the signals to determine and/or adjust the SNR may result in additional power consumption for the patient monitor.

Additionally, the patient monitor may utilize a timing cycle, which may include alternating between emitting periods and dark periods. As used herein, a dark period is an interval in which no light is emitted from the emitter. The patient monitor may drive the emitter for a duration that allows the current of the emitter to settle (i.e., stabilize at a desired current level) and the photo-signals (e.g., the light detected by a detector) to settle before measuring the photo-signals (i.e., the light detected by a detector). Generally, the amplitude of the photo-signals may vary at the beginning of the emitting periods due to various factors, such as the opacity of the tissue of the patient and/or the distance between the emitter and the detector. This method of operation may enable the patient monitor to obtain more accurate measurements of the photo-signals. However, a longer emitting period may increase the power need of the medical sensor system. Additionally, for pulse oximetry systems, the patient monitor generally acquires one measurement per emitting period because the sampling rate may be limited due to an analog-to-digital converter with a slow response time. As used herein, a sample is a subset of all of the measurements that could be derived from a signal (e.g., photo-signals or a photocurrent) generated by a medical sensor system. Accordingly, acquiring a greater number of measurements may yield a higher SNR and enable a more accurate determination of the sample. As such, typical pulse oximetry systems may drive the emitter more frequently (e.g., shorter dark periods) to obtain more measurements for the determination of the sample. However, an increase in overall emitting time may increase the power consumption of the system.

Provided herein are techniques to reduce the power consumption of a patient monitor. For example, rather than calculating the SNR to determine an appropriate current level to drive an emitter, the present embodiments may include sampling one or more operating parameters of a medical sensor at predetermined times. The one or more operating parameters may be compared to a predetermined range of values appropriate for normal operating ranges and then may be adjusted accordingly. The one or more operating parameters may include, for example, the current and/or voltage level of the emitter during emitting periods. For example, if the patient monitor determines that the current of the emitter during the emitting periods exceeds a maximum threshold, the patient monitor may decrease the current of the emitter.

Additionally, the present embodiments include a power-reducing timing cycle for generating, sampling, and digitizing photo-signals of the medical sensor system. As used herein, a power-reducing timing cycle is defined as a sequence of interleaved emitting periods and dark periods executed by a patient monitor, in which the dark periods occur for a longer duration than the emitting periods. Operating the medical sensor system using the power-reducing timing cycle may enable the medical sensor system to reduce the overall power consumption, as compared to embodiments in which the power-reducing cycle is not employed. For example, in certain embodiments, the dark periods may be approximately five times longer than the emitting periods, which may advantageously reduce the power consumption of the emitter to less than four percent of the power consumption for timing cycles in which the emitting periods are approximately equal to the dark periods. However, the power-reducing timing cycle may reduce the number of photo-signals generated and thus, may reduce the SNR. Accordingly, the in certain embodiments, the patient monitor may acquire multiple measurements of photo-signals per emitter excitation period, rather than one measurement per emitter excitation period, to obtain a desired SNR.

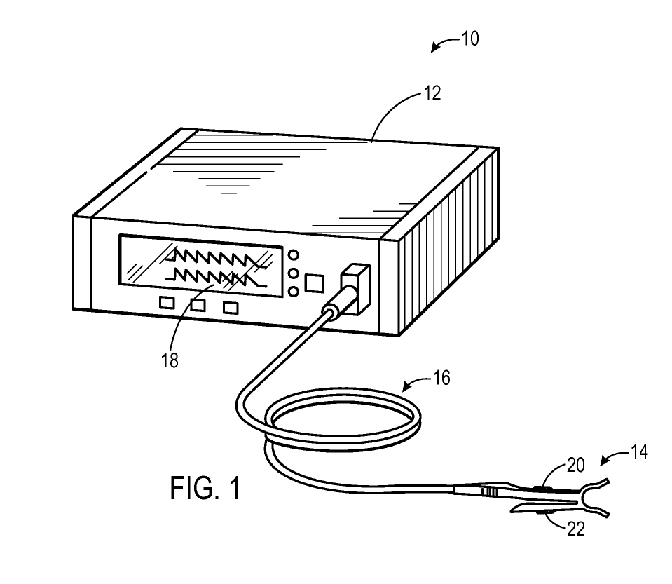

With the foregoing in mind, FIG. 1 illustrates an embodiment of a non-invasive medical sensor system 10 having a patient monitor 12 and a medical sensor 14. Although the embodiment of the medical sensor system 10 illustrated in FIG. 1 relates to a patient monitor configured to obtain pulse oximetry measurements (e.g., blood oxygen saturation or pulse rate), the medical sensor system 10 may be configured to obtain a variety of physiological measurements. For example, the medical sensor system 10 may, additionally or alternatively, measure water fraction of tissue or perform other non-invasive medical monitoring techniques.

The patient monitor 12 may exchange signals with the medical sensor 14 via a communication cable 16. In other embodiments, the patient monitor 12 may wirelessly communicate with the medical sensor 14. The patient monitor 12 may include a display 18 and various monitoring and control features. In certain embodiments, the patient monitor 12 may include a processor that may determine a physiological parameter of a patient based on signals obtained from the medical sensor 14. Indeed, in the presently illustrated embodiment of the medical sensor system 10, the medical sensor 14 is a pulse oximetry sensor that may non-invasively obtain pulse oximetry data from a patient. In other embodiments, the medical sensor 14 may represent any other suitable non-invasive optical sensor.

The medical sensor 14 may attach to pulsatile patient tissue (e.g., a patient's finger, ear, forehead, or toe). In the illustrated embodiment, the medical sensor 14 is configured to attach to a finger. An emitter 20 and a detector 22 of the medical sensor 14 may operate to generate non-invasive pulse oximetry data for use by the patient monitor 12. In particular, the emitter 20 may transmit light at certain wavelengths into the tissue and the detector 22 may receive the light after it has passed through or is reflected by the tissue. The amount of light and/or certain characteristics of light waves passing through or reflected by the tissue may vary in accordance with changing amounts of blood contingents in the tissue, as well as related light absorption and/or scattering.

The emitter 20 may emit light from one or more light emitting diodes (LEDs) or other suitable light sources into the pulsatile tissue. The light that is reflected or transmitted through the tissue may be detected using the detector 22 (e.g., a photodiode). The detector 22 may generate a photocurrent proportional to the amount of detected light, and the photocurrent may be transmitted through the cable 16 to the patient monitor 12. As described in greater detail below, the patient monitor 12 may convert the photocurrent from the detector 22 into a voltage signal that may be analyzed to determine certain physiological characteristics of the patient (e.g., SpO₂).

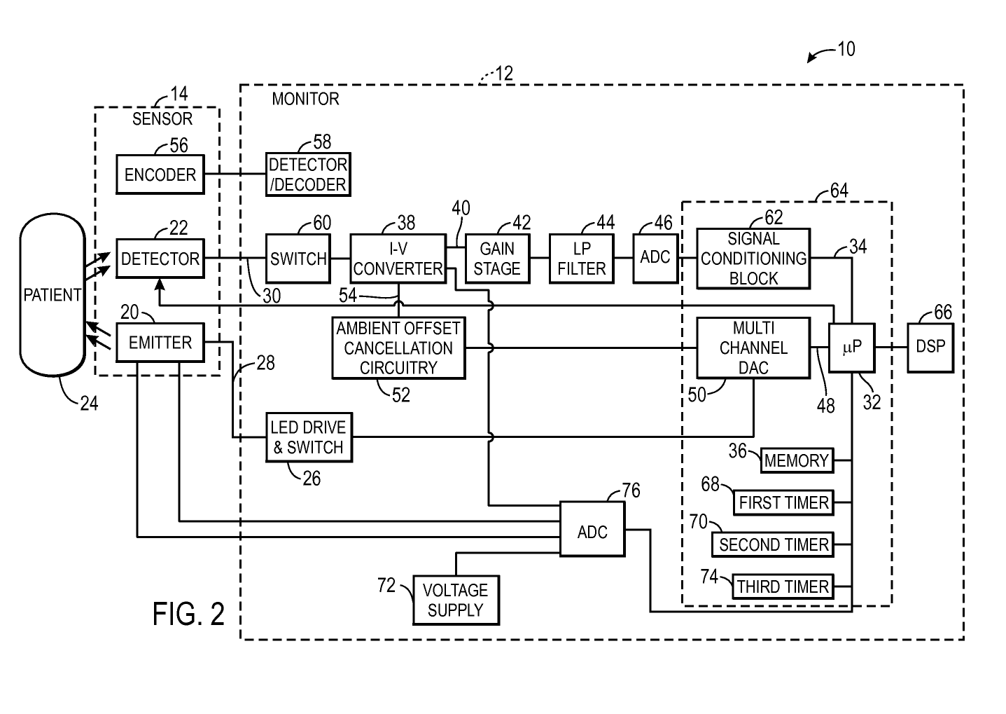

As illustrated in FIG. 2, the emitter 20 may emit light into a patient 24, which may be reflected by or transmitted through the patient 24 and detected by the detector 22. An LED drive and/or switch 26 (e.g., drive circuitry) may generate LED driving signals (e.g., LED current signals 28) to cause the LEDs of the emitter 20 to emit the light into the patient 24. In some embodiments, the LEDs of the emitter 20 may emit one or more different wavelengths of light. In certain embodiments, the LED current signals 28 may include red wavelengths of between approximately 600 nm and 700 nm and/or infrared wavelengths of between approximately 800 nm and 1000 nm. In other embodiments, the LED current signals 28 may include a red wavelength of between approximately 620 nm and 700 nm (e.g., 660 nm), a far red wavelength of between approximately 690 nm and 770 nm (e.g., 730 nm), and an infrared wavelength of between approximately 860 nm and 940 nm (e.g., 900 nm). Other wavelengths may include, for example, wavelengths of between approximately 500 nm and 600 nm and/or 1000 nm and 1100 nm. Regardless of the number and wavelength of LEDs driven by the LED drive and/or switch 26, the LED current signals 28 may include at least one dark period during which no LEDs of the emitter 20 are being driven (i.e., LED current signals 28 are not provided to the emitter 20). The at least one dark period may enable a reduction in power consumption of the medical sensor system 10 compared to a system that constantly drives the emitter 20.

The detector 22 may detect the emitted light that passes through or is reflected by the tissue of the patient 24. In response to the light, the detector 22 may generate a photocurrent signal 30 that varies depending on the amount and wavelength of light emitted by the emitter 20 and the various physiological characteristics of the patient 24. In addition, the detector 22 may also generate a component of the photocurrent signal 30 in response to ambient light near the patient 24 (e.g., room lighting or light from windows).

In certain embodiments, the photocurrent signal 30 may be transmitted to the monitor 12. The monitor 12 may include data processing circuitry (such as one or more processors 32 or application specific integrated circuits (ASICS)) coupled to an internal bus 34 for processing the photocurrent signal 30. The monitor 12 may also include at least one memory 36 for storing coded instructions and/or algorithms that may be accessed and executed by the processor 32. The memory 36 may be any suitable computer-readable storage memory, such as a RAM, a ROM, and/or a mass storage device. The memory 36 may include a plurality of components such as one or more electronic components, hardware components, and/or computer software components. In certain embodiments, the memory 36 may be non-transitory and tangible. The memory 36 may employ, for example, one or more of a magnetic, electrical, optical, biological, and/or atomic data storage medium. Further, the memory 36 may take the form of, for example, floppy disks, magnetic tapes, CD-ROMs, DVD-ROMs, hard disk drives, and/or solid-state or electronic memory. Other forms of non-transitory, tangible computer readable storage media not listed may be employed with the disclosed embodiments.

A current-to-voltage (I-V) converter 38 may also be coupled to the internal bus 34 and may convert the photocurrent 30 from the detector 22 into an output voltage signal 40. In certain embodiments, the I-V converter 38 may also be configured to reject low frequency common mode signals (e.g., transient interference generated from a cable connecting the sensor 14 to the monitor 12). The output voltage signal 40 may be provided to a gain stage 42. The gain stage 42 may be an amplifier having both a unity gain and a multiplier gain. The microprocessor 32, which will be discussed in more detail below, may control the gain stage 42, and more specifically, may be configured to select the unity gain or the multiplier gain. In certain embodiments, the multiplier gain of the gain stage 42 may be desirable to increase the signal level, while reducing the level of the LED current signals 28 to reduce power consumption. However, applying the multiplier gain may not increase the SNR. In certain embodiments, a low pass (LP) filter 44 may filter the output voltage signal 40. In one embodiment, the LP filter 44 may have a bandwidth configured to achieve anti-aliasing of high frequency components of the output voltage signal 40. After filtering, an analog-to-digital converter (ADC) 46 may digitize the output voltage signal 40, and after digitizing, the microprocessor 32 may receive the output voltage signal 40.

As mentioned above, a portion of the photocurrent 30 from the detector 22 may be due to ambient light detected by the detector 22. If the I-V converter 38 simply converted the entire photocurrent signal 30 into an output voltage signal 40 without accounting for components of the photocurrent signal 30 that correspond to the ambient light, the output voltage signal 40 could saturate and become distorted. In some embodiments, the gain of the I-V converter 38 could be reduced to prevent such saturation, improving the SNR. To prevent signal distortion resulting from output voltage signal 40 saturation and/or to improve the I-V converter 38 SNR, the patient monitor 12 may include certain components and/or circuitry to cancel the effect of ambient light on the photocurrent 30.

For example, in certain embodiments, the microprocessor 32 may sample the output voltage signal 40 (e.g., after being filtered in the LP filter 44 and digitized by the ADC 46) during dark periods when the emitter 20 is not emitting any light. During such dark periods, substantially all of the photocurrent signal 30, and the corresponding voltage signal 40, may be attributable to ambient light. The resulting output voltage signal 40 obtained during dark periods may be used to cancel out the ambient light component of the photocurrent signal 30 in the I-V converter 38 during emitting periods. In one embodiment, the microprocessor 32 may determine a feedback signal 48 that, when provided to the I-V converter 38, causes the I-V converter 38 to largely exclude the ambient light component of the photocurrent 30 in a variety of manners, as discussed below.

In one embodiment, a digital-to-analog converter (DAC) 50 may convert the digital value of the feedback signal 48 to an analog value, and may provide the analog value to ambient offset cancellation circuitry 52. The ambient offset cancellation circuitry 52 may generate a corresponding feedback signal 54 that may be provided to the I-V converter 38. As noted above, the feedback signal 54 may cause the output voltage 40 of the I-V converter 38 to include substantially only emitted light that has passed through the patient 24 and detected by the detector 22. Also, the microprocessor 32 may control the LED drive and/or switch 26 via the DAC 50. In one embodiment, the DAC 50 may be a multi-channel DAC to enable the provision of signals to the LED drive and/or switch 26 and the ambient offset cancellation circuitry 52.

Additionally, in certain embodiments, the medical sensor 14 may include an encoder 56 that may provide signals to the microprocessor 32 related to characteristics of the medical sensor 14, which may assist the microprocessor 32 in determining the feedback signal 48 to provide to the ambient offset cancellation circuitry 52. For example, the encoder 56 may indicate a propensity of the medical sensor 14 to detect ambient light. In certain embodiments, the encoder 56 may provide an offset voltage representing a typical ambient light voltage, which may serve as an initial starting voltage of the feedback signal 48. Additionally, the encoder 56 may provide signals indicative of the wavelength of one or more light sources of the emitter 20, which may allow for the selection of appropriate calibration coefficients for calculating a physical parameter such as blood oxygen saturation. The encoder 56 may, for instance, be a coded resistor, EEPROM or other coding devices (such as a capacitor, inductor, PROM, RFID, parallel resident currents, or a colorimetric indicator) that may provide a signal to the microprocessor 32 related to the characteristics of the medical sensor 14 to enable the microprocessor 32 to determine the appropriate calibration characteristics of the medical sensor 14. Further, the monitor 12 may include a reader/decoder 58 that may read and/or decode information from the encoder 56 to provide the processor 32 with information about the medical sensor 14. In some embodiments, the encoder 56 and/or the reader/decoder 58 may not be present.

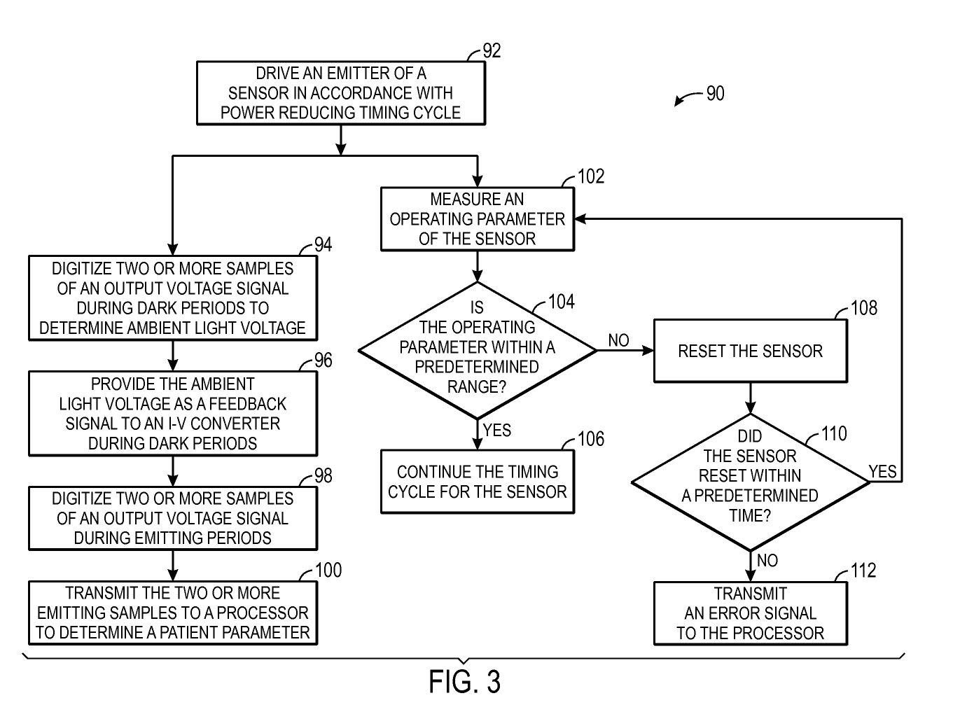

As described above, providing the feedback signal 54 to the I-V converter 38 may improve the SNR of the output voltage signal 40 by removing components resulting from ambient light. As a result of the improved SNR, the microprocessor 32 may be configured to control the LED drive and/or switch 26 in accordance with a power reduction scheme, which will be described in more detail below with respect to FIGS. 3-5. For example, in one embodiment, the LED drive and/or switch 26 may emit lower current signals 28, which may enable the medical sensor system 10 to operate at a lower power level than if this embodiment were not employed. Additionally or alternatively, the microprocessor 32 may be configured to implement a power-reducing timing cycle in which the dark periods are longer than the emitting periods. Thus, the power-reducing timing cycle effectively reduces the overall power provided to the emitter 20.

To further reduce the power consumption of the medical sensor system 10, the patient monitor 12 may include a switch 60 that is controlled by the microprocessor 32 to control the gating-in of the photocurrent 30 from the detector 22. During the dark periods of the power-reducing timing cycle, the photocurrent 28 may not contain significant information for determining a physiological parameter of the patient 24. Thus, it may be desirable to control the gating-in of the photocurrent 30 such that none or only a portion of the photocurrent 30 generated during dark periods is received by the I-V converter 38 from the detector 22. Specifically, the portion of the photocurrent 30 transferred to the I-V converter 38 by the switch 60 may be selected such that a desired number of measurements of the photocurrent 30 may be digitized and analyzed to determine the feedback signal 54, while the remainder of the photocurrent 30 generated during the dark period may be discharged to ground by the switch 60. Thus, the switch 60 may reduce the overall amount of processing power. Alternatively, the I-V converter 38 may continuously receive the photocurrent 30, and the microprocessor 32 may process only relevant portions of the output voltage signal 40 to reduce the power consumption.

As will be appreciated, reducing the emitter 20 excitation time may reduce the number of photo-signals generated. As such, the output voltage signal 40 may correspond to fewer photo-signals. Accordingly, it may be desirable to provide additional techniques to improve the SNR of the output voltage signal 40 to enable more accurate calculations of one or more physiological characteristics of the patient 24. For example, digitizing two or more measurements of the output voltage signal 40 per emitting period rather than one measurement, which may be typical for pulse oximetry systems, increases the signal level and thus, improves the SNR.

Thus, in certain embodiments, the ADC 46 may be configured to digitize two or more measurements of the output voltage signal 40 per emitting period. Additionally, the ADC 46 may digitize a corresponding number of measurements of the output voltage signal 40 during the dark periods, to enable the microprocessor 32 to determine the feedback signal 48. Specifically, the ADC 46 may generate red measurements, post-red dark measurements, IR measurements, and post-IR dark measurements, where each measurement corresponds to the signals obtained during the respective period. More specifically, the post-red dark measurements correspond to signals obtained in the dark period following a red LED emitting period and similarly, the post-IR dark measurements correspond to signals obtained during the dark period following an IR LED emitting period. In one embodiment, the post-red and post-IR dark measurements may be obtained following the red and IR emitting periods, respectively. While the ADC 46 may digitize any number of measurements per emitting period, in certain embodiments, the ADC 46 may digitize between one measurement and twelve measurements per period, two measurements and eight measurements per period, or three measurements and six measurements per period. In one embodiment, the ADC 46 may digitize four measurements per period. For each measurement, the ADC 46 may sample and hold the output voltage signal 40 for a short time to enable the ADC 46 to convert (i.e., digitize) the sampled output voltage signal 40. This time may be defined as the conversion time. In certain embodiments, the conversion time may be between 1 μ seconds and 5 μ seconds. In one embodiment, the conversion time may be approximately 2 μ seconds. As will be appreciated, a conversion time of approximately 2 μ seconds may result in a conversion speed of approximately one conversion every 2 μ seconds. Additionally, in certain embodiments, the ADC 46 may have a resolution of at least 16 bits to obtain more accurate measurements of the output voltage signal 40.

After being digitized by the ADC 46, the measurements may undergo initial processing before the calculation of one of more physiological parameters. In one embodiment, the measurements may be received by the microprocessor 32 from the ADC46. Certain embodiments of the monitor 12, however, may include a signal conditioning block 62, which may receive the measurements from the ADC 46, perform signal conditioning on the measurements, and send the measurements to the microprocessor 32. In one embodiment, the signal conditioning block 62 and the microprocessor 32 may be elements of a single-chip microcontroller 64, which will be discussed in more detail below. In certain embodiments, the signal conditioning block 62 may include a demodulator. The demodulator may interpret the received measurements as, for example, corresponding to light in either the red or the infrared spectrum. In certain embodiments, the signal conditioning block 62 may compare each measurement to a threshold value to determine if the measurement is within normal range. A measurement outside of its normal range may indicate that the LED current signals 28 are higher or lower than a desired current level, which may reduce the efficiency of the medical sensor system 10. If a measurement exceeds its corresponding threshold value, the signal conditioning block 62 may notify the microprocessor 32, which may determine whether the LED current signals 28 should be adjusted. Additionally, the signal conditioning block 62 may subtract the dark measurements from the corresponding LED measurements. However, in some embodiments, the ambient light components of the LED measurements may have been previously offset via the feedback signal 54. Regardless of the method for offsetting the ambient light, the signal conditioning block 62 may transfer the red and IR LED measurements having a reduced amount of ambient light components (e.g., substantially no ambient light components) to the microprocessor 32.

In some embodiments, the microprocessor 32 may receive the conditioned measurements from the signal conditioning block 62 and may transfer the conditioned measurements to another microprocessor. For example, the microprocessor 32 may transfer the conditioned measurements and/or other data to a digital signal processor (DSP) 66, which may use various algorithms to determine certain physiological parameters of the patient 24. The output rate of measurements from the microprocessor 32 and/or the input rate of measurements to the DSP 66 may differ from the input rate of measurements to the signal conditioning block 62. Accordingly, the signal conditioning block 62 may down-sample the measurements to match the appropriate output or input rate of the microprocessor 32 or the DSP 66, respectively. For example, the signal conditioning block 62 may average the red LED measurements and the IR LED measurements so that one red LED measurement and one IR LED measurement are output by the microprocessor 32.

The microprocessor 32 and/or the single-chip microcontroller 64 may control the timing of the medical sensor system 10 and may interface with the DSP 66. Accordingly, the microprocessor 32 and/or the single-chip microcontroller 64 may include one or more features for controlling the operation of the medical sensor 14 and/or the processing of the output voltage signal 40. For example, the one or more features may include multiple internal programmable timers, a serial peripheral interface (SPI), a flash memory, and a random-access memory (RAM), which may be a static RAM (SRAM). The timers may reduce the overall processing and thus, reduce the power consumption as compared to embodiments in which the timers are not employed. That is, once a timer has been programmed, it may operate without additional software intervention (e.g., from the microprocessor 32). Furthermore, the timers may reduce timing latencies, which may occur when using software to control the timing (e.g., via interrupt service routines). In one embodiment, the timers may be high-resolution 16 bit timers. Specifically, the resolution of the timers may be between 0.1 and 0.5 μ seconds. Furthermore, in certain embodiments the timers may have an interrupt latency that is less than 5 μ seconds, less than 3 μ seconds, or less than 1 μ second.

In certain embodiments, a first timer 68 may control turning on of the red and IR LEDs of the emitter 20. In one embodiment, the first timer 68 may also control the turning off of the red and IR LEDs. The first timer 68 may be programmed with a desired timing cycle using data from the microprocessor 32. For example, the data from the microprocessor 32 may be data relating to a pulse of the patient 24, which may be used to synchronize the emitter 20 excitation. In certain embodiments, the data may be received by the microprocessor 32 from an external source, such as an electrocardiography (ECG) sensor. The synchronized timing may enhance pulse signal identification in the DSP 66. The timer 68, which may reduce timing latencies as compared to software intervention, may generate a more precise timing cycle and may result in a more accurate output voltage signal 40 as compared to embodiments in which the timer is not employed.

A second timer 70, or alternatively, the first timer 68, may be programmed to control the timing of measurement acquisitions in the ADC 46. That is, the second timer 70 may control when the ADC 46 digitizes the output voltage signal 40 and when the resulting measurement is transferred to the signal conditioning block 62 or the microprocessor 32. The second timer 70 may also be programmed by the microprocessor 32. The timing of the measurement acquisitions may be selected such that the current of the emitter 20 and the photo-signals have sufficient time to settle before measurements are acquired. For example, to enable more accurate measurements, the second timer 70 may be programmed to coincide with the first timer 68 such that the output voltage signal 40 is digitized after the LED current signals 28 have settled. Similar to the first timer 68, the second timer 70 may result in more accurate measurements, as the second timer 70 may reduce timing latencies, as well as a reduction in power consumption.

As noted above, another technique to reduce the power consumption of the medical sensor system 10 may involve measuring one or more operating parameters of the medical sensor 14 at predetermined times to determine whether the operating parameters are within a normal operating range, rather than analyzing the signals to determine the SNR. That is, rather than calculating the SNR to determine whether one or more operating parameters should be adjusted to achieve a desired SNR, the monitor 12 may determine whether the operating parameters are within the normal range and may determine that the output voltage signal 40 likely has a sufficient SNR if the operating parameters are each within the normal range. For example, the one or more operating parameters may include the current and/or voltage of the emitter 20 during the red and IR LED emitting periods, the current of the emitter 20 during the dark periods (e.g., just after the LED is turned off), the voltage of the detector 22, and/or the voltage of a voltage supply 72 provided to the medical sensor 14.

Accordingly, a third timer 74 of the microprocessor 32 and/or of the single-chip microcontroller 64, which may be programmed with timing information to control the measurement acquisition of the operating parameters. Specifically, the third timer 74 may control the measurement acquisition from one or more analog-to-digital converters (ADC) 76 of the patient monitor 12. In certain embodiments, the analog-to-digital converter 76 may be a multi-channel ADC 72 that is configured to digitize measurements corresponding to more than one operating parameter. For example, one channel of the multi-channel ADC 76 may correspond to the acquisition of the current of the emitter 20, while another channel may correspond to the acquisition of the voltage of the detector 22. Alternatively, the patient monitor 12 may include an ADC 76 that is specific for each desired operating parameter.

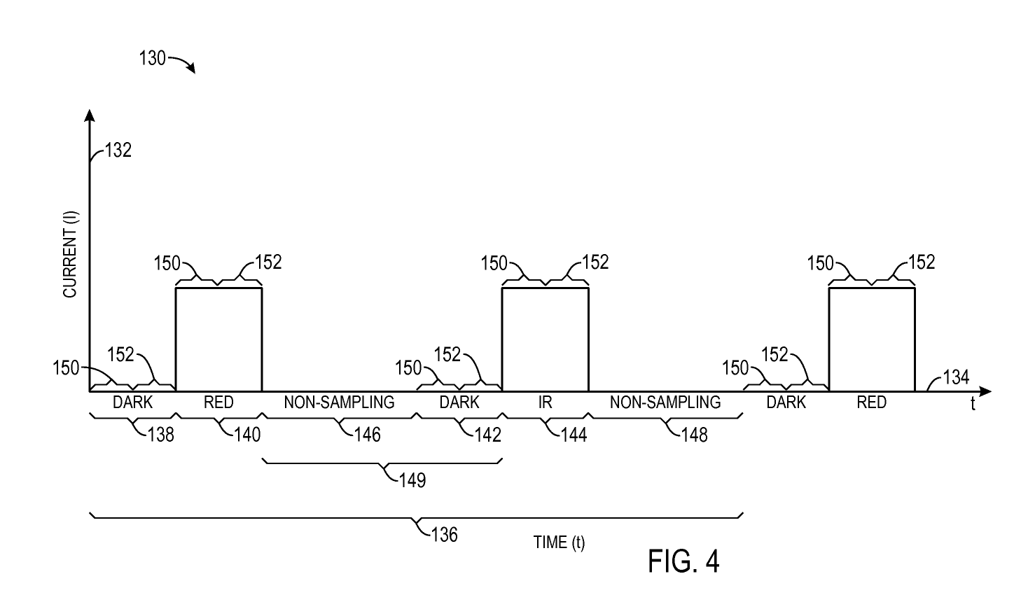

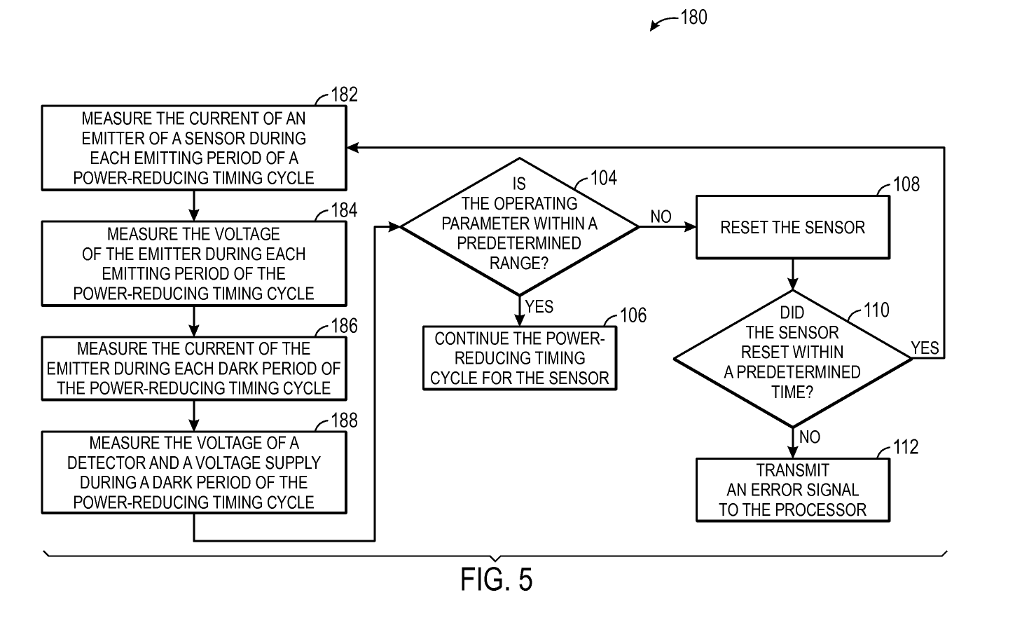

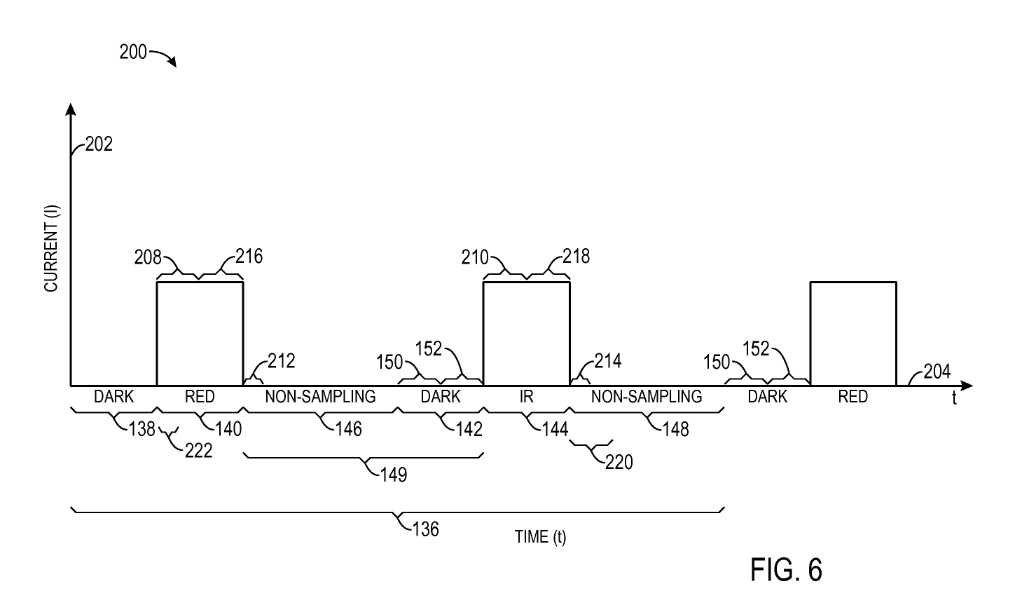

As described in detail above, the medical sensor system 10 discussed with respect to FIGS. 1 and 2 may reduce the overall power consumption of the medical sensor system 10 by implementing a power-reducing timing cycle. In particular, the power-reducing timing cycle may include emitting periods and dark periods, where the dark periods occur for a longer duration as compared to the emitting periods, and may include measuring the operating parameters of the medical sensor 14, rather than calculating the SNR. Indeed, the present embodiments provide various methods, discussed in detail below, for reducing the power consumption of the medical sensor system 10 in accordance with the embodiments discussed above. For example, FIG. 3 illustrates an embodiment of a method of operation for the medical sensor system 10 including implementing a power-reducing timing cycle for the medical sensor system 10. FIG. 4 illustrates a graph (e.g., a timing diagram) of current pulses (e.g., the LED current signals 28) provided to the emitter 20 of the medical sensor 14 for measuring the output voltage signal 40 in accordance with the method of FIG. 3. Additionally, as the power-reducing cycle also may include measuring one or more operating parameters of the medical sensor 14, FIG. 5 illustrates an embodiment of a method for measuring the operating parameters of the medical sensor 14. FIG. 6 illustrates a graph (e.g., a timing diagram) of current pulses (e.g., the LED current signals 28) for measuring the operating parameters of the medical sensor 14 in accordance with the method discussed with respect to FIG. 5.

Referring now to FIG. 3, an embodiment of a method 90 for operating the medical sensor system 10 to determine physiological parameters of the patient 24 is illustrated. Certain steps of the method 90 may be performed by a processor, or a processor-based device such as the patient monitor 12 that includes instructions for implementing certain steps of the method 90. Additionally, the instructions for implementing certain steps of the method 90 may be stored as coded instructions and/or algorithms in the memory 36 and may be accessed and executed by the processor 32. The method 90 includes driving the emitter 20 of the medical sensor 14 with the LED drive and/or switch 26 in accordance with a power-reducing timing cycle (block 92). As described above, a power-reducing timing cycle includes emitting periods and dark periods, where the dark periods occur for a longer duration as compared to the emitting periods. A longer dark period may be advantageous to reduce the power consumption of the emitter 20. However, as discussed in detail above, the longer dark periods may reduce the SNR. Accordingly, the present embodiments may provide a balance between a desired reduction in power and a desired SNR. Specifically, the present embodiments balance the duration of the dark periods with the duration of the emitting periods and the number of measurements digitized per emitting period to achieve the SNR. For example, in certain embodiments, the dark periods may at least twice as long as the emitting periods. In one embodiment, the dark periods may be five times longer than the emitting periods.

As noted above, during the dark periods substantially all of the light detected by the detector 22 may be ambient light. Thus, during one or more such dark periods, the microprocessor 32 may cause the ambient offset cancellation circuitry 52 to tie the feedback signal 54 to ground, and the ADC 46 may digitize two or more measurements of the resulting output voltage signal 40. The output voltage signal 40 may be analyzed by the microprocessor 32 to determine the ambient light voltage (block 94). That is, the output voltage signal 40 obtained during the dark periods while the feedback signal 54 is set to a ground voltage may represent a baseline ambient light voltage of the photocurrent 30. Thus, the microprocessor 32 may provide the determined baseline ambient light voltage as the feedback signal 54 during emitting periods (e.g., a red period and/or an IR period) (block 96). If multiple dark periods are considered, the microprocessor 32 may average the baseline ambient voltage obtained during the multiple dark periods. Alternatively, the microprocessor 32 may determine a baseline ambient light voltage for each dark period.

Thereafter, during the emitting periods, the microprocessor 32 may cause the ambient offset cancellation circuitry 52 to provide the baseline ambient light voltage as the feedback signal 54 to the I-V converter 38. Accordingly, the ADC 46 may digitize two or more measurements of the resulting output voltage signal 40, which may substantially exclude ambient light noise, during both emitting periods (block 98). In certain embodiments, the emitter 20 may include a red LED and an IR LED, and as such, the ADC 46 may digitize two or more measurements during a red emitting period and two or more measurements during an IR emitting period. Furthermore, as noted above, a greater number of measurements for each emitting period may increase the SNR. Accordingly, in certain embodiments, it may be desirable to digitize between three and six measurements per emitting period. In one embodiment, the ADC 46 may digitize four measurements per emitting period. It should be appreciated that the number of measurements digitized during the dark periods may be adjusted to match the number of measurements digitized during the emitting periods.

Next, the two or more measurements obtained during the emitting periods may be transmitted to a processor (e.g., the DSP 66) to determine one or more physiological parameters of the patient 24 (block 100). As described above, the ADC 46 may first transmit the measurements to the signal conditioning block 60 for initial processing before the measurements are received by the processor 32. For example, the two or more measurements may be down-sampled (e.g., averaged) to a rate suitable to be output by the microprocessor 32 and/or input to the DSP 66.

Additionally, the method 90 may include measuring an operating parameter of the medical sensor 14 (block 102). As will be discussed in more detail below with respect to FIGS. 5 and 6, the patient monitor 12 may measure one or more operating parameters of the medical sensor 14, which may be measured at different times. As discussed above, the one or more operating parameters may include the current and/or voltage of the emitter 20 during the red and IR LED emitting periods, the current of the emitter 20 during the dark periods (e.g., just after the LED is turned off), the voltage of the detector 22, and/or the voltage of a voltage supply 72 provided to the medical sensor 14. The method 90 may also include determining whether the operating parameter is within a predetermined range (block 104). Specifically, the patient monitor 12 may compare the measurement of the operating parameter to a predetermined upper and/or a lower threshold, which may be specific for the operating parameter and/or for the medical sensor 14. In certain embodiments, the encoder 56 of the medical sensor 14 may include stored data related to the predetermined upper and/or lower threshold for one or more of the operating parameters, which may be read by the microprocessor 32. Accordingly, if the patient monitor 12 determines that the measurement of the operating parameter is within the predetermined range, the patient monitor 12 may continue implementing the power-reducing timing cycle for the medical sensor 14 (block 106).

However, if the operating parameter is not within the predetermined range, the method 90 may include resetting the medical sensor 14 (block 108). Specifically, the patient monitor 12 may momentarily cease a supply of power to the medical sensor 14 to facilitate resetting. The method 90 may further include determining whether the medical sensor 14 reset within a predetermined time (block 110). The microprocessor 32 may determine that the medical sensor 14 successfully reset if the operating parameters of the medical sensor 14 (e.g., the current and/or the voltage of the emitter 20) return within their corresponding predetermined threshold ranges after the medical sensor 14 is momentarily disconnected from power. Failing to reset within the predetermined time may indicate a faulty sensor. Accordingly, the patient monitor 12 may transmit an error signal to the DSP 66 in response to determining that the medical sensor 14 has failed to reset within the predetermined time (block 112). Additionally, the DSP 66 may analyze the error signal and display an error message to a user on the display 18. For example, the error message may indicate to a user that the medical sensor 14 should be replaced.

In other embodiments, prior to, or instead of, resetting the medical sensor 14 (block 108), the microprocessor 32 may adjust the LED current signals 28 based at least in part upon the comparison of the measured operating parameter to its respective threshold range. For example, if the microprocessor 32 determines that the current of the emitter 20 is less than the predetermined lower threshold, the microprocessor 32 may increase the current of the LED current signals 28. The analog-to-digital converter 76 may digitize a second measurement of the operating parameter after the microprocessor 32 adjusts the LED current signals 28, and the microprocessor 32 may compare the second measurement to the respective threshold range. If the operating parameter is still not within the predetermined range, the microprocessor 32 may cause the medical sensor 14 to reset (block 108). However, if the operating parameter is within the predetermined range, the patient monitor 12 may continue implementing the power-reducing timing cycle for the medical sensor 14 (block 106).

FIG. 4 illustrates a timing diagram 130 of current pulses (e.g., the LED current signals 28) provided to the emitter 20 of the medical sensor 14 over time and the time points for measuring the resulting output voltage signal 40 in accordance with an embodiment of the power-reducing timing cycle. The timing diagram 130 depicts the current 132 provided to a red LED and an IR LED of the emitter 20 as a function of time 134). The timing diagram 130 also depicts a multiplexing period 136 of the power-reducing timing cycle, which includes a first dark period 138, a first red period 140, a second dark period 142, and a first IR period 144. The first and the second dark periods 138 and 142 are periods of the multiplexing period 136 when the emitter 20 does not emit light into the patient 24 and the patient monitor 12 measures the photocurrent 30 to determine the feedback signal 54. Thus, the first and the second dark periods 138 and 142 may be sampling periods.

While the multiplexing period 136 may be any suitable duration, in accordance with certain embodiments, the multiplexing period 136 may be between 2000 and 4000 μ seconds or between 2500 and 3500 μ seconds. In one embodiment, the multiplexing period 136 may have a duration of approximately 3200 μ seconds, and the patient monitor 12 may sample the output voltage signal 40 at about 311.25 Hz periodically throughout the multiplexing period 136. A typical pulse oximeter may sample a signal output from a detector at about 1211 Hz periodically throughout a multiplexing period. As such, the present disclosure may provide a significant reduction in the sampling frequency to reduce the power consumption of the medical sensor system 10. For example, reducing the sampling frequency from 1211 Hz to about 311.25 Hz may reduce the power need of the medical sensor system 10 by at least a factor of 32.

The multiplexing period 136 also includes a first non-sampling period 146 following the first emitting period (e.g., the first red period 140), and a second non-sampling period 148 following the second emitting period (e.g., the first IR period 144). The first and the second non-sampling periods 146 and 148, however, are periods of the multiplexing cycle 136 when the emitter 20 does not emit light into the patient 24 and the patient monitor 12 does not receive and/or process the photocurrent 30. That is, in certain embodiments, the microprocessor 32 may synchronize the operation of the detector 22 with the multiplexing period 136 such that the detector 22 may not receive power during the first and the second non-sampling periods 146 and 148. Thus, in certain embodiments, the detector 22 may not generate the photocurrent 30 during the first and the second non-sampling periods 146 and 148. In other embodiments, the detector 22 may receive power, and the switch 60 (FIG. 2) may discharge the photocurrent 30 obtained during the first and the second non-sampling periods 146 and 148 to ground. In one embodiment, the microprocessor 32 may receive, but not process, the resulting output voltage signal 40. As such, the first and the second non-sampling periods 146 and 148 may be desirable to reduce the overall power consumption of the medical sensor system 10. Indeed, power requirements of the medical sensor system 10 during the first and the second non-sampling periods 146 and 148 may be negligible. Accordingly, the multiplexing period 136 includes a dark period 149 that includes a dark sampling period and a dark non-sampling period between the emitting periods, and the dark periods 149 may be longer than the emitting periods. Thus, implementing the power-reducing timing cycle with the dark periods 149 may enable the medical sensor system 10 to operate using less power than embodiments in which the dark periods 149 are not employed.

Accordingly, the duration of each period of the multiplexing period 136 may be selected to reduce the LED current signals 28 provided to the emitter 20 (e.g., reduce the emitting time) and to maximize the SNR (e.g., obtain a greater number of measurements). In certain embodiments, the duration of each dark, red, and/or IR period may be selected to include a measurement delay period 150 and a measurement period 152. The measurement delay period 150 may be desirable to allow the photocurrent 36 and the current of the emitter 20 to settle (e.g., stabilize at a desired current level) before acquiring measurements. Generally, the amplitude of the photocurrent 30 may vary at the beginning of the emitting periods due to various factors, such as the opacity of the tissue of the patient 24 and/or the distance between the emitter 20 and the detector 22. Additionally, the current of the emitter 20 may fluctuate when the LED current signals 28 are first provided to the emitter 20 before stabilizing at a desired current level. Thus, the measurement delay period 150 may enable a more accurate measurement of the photocurrent 30. During the measurement delay period 150, the patient monitor 12 may receive and process the photocurrent 30, but may not acquire measurements of the processed signal to be used in the calculation of a physiological parameter. In certain embodiments, the measurement delay period 150 may be between 50 and 250 μ seconds or between 100 and 200 μ seconds. In one embodiment, the measurement delay period 150 may be 160 μ seconds. Additionally, the duration of the measurement delay period 150 may be between approximately 1 percent and 15 percent, 2 percent and 12 percent, or 3 percent and 10 percent of the duration of the multiplexing period 136. In one embodiment, the duration of the measurement delay period 150 may be approximately 5 percent of the duration of the multiplexing period 136.

The measurement period 152 may occur after the measurement delay period 150. During the measurement period 152, the ADC 46 may digitize two or more measurements of the output voltage signal 40, and the microprocessor 32 and/or the signal conditioning block 60 may read the digitized measurements from the ADC 46. In other words, during the measurement period 152 more than one measurement of the output voltage signal 40 may be obtained. Accordingly, the duration of the measurement period 152 may be adjusted based upon the conversion speed of the ADC 46, the time to read the measurements from the ADC 46, and the number of measurements desired. For example, the measurement period 152 may be between 50 and 250 μ seconds or between 75 and 150 μ seconds. In certain embodiments, the duration of the measurement period 152 may be between approximately 1 percent and 12 percent or 2 percent and 10 percent of the duration of the multiplexing period 136. In one embodiment, the duration of the measurement period 152 may be approximately three percent of the duration of the multiplexing period 136. It should be noted that the emitting periods (e.g., the first red and the first IR periods 140 and 144) may include an additional delay period following the corresponding measurement period 152, which may occur as the corresponding LED turns off. For example, an LED may take between 20 and 40 μ seconds to completely stop emitting light. Accordingly, as the amount of light emitted into the patient 24 may fluctuate during this time, it may not be desirable to continue measuring the output voltage signal 152.

In a similar manner to the emitting periods, the duration of the first and the second non-sampling periods 146 and 148 may be selected to maximize power reduction and to obtain a desired SNR. Specifically, a longer non-sampling period may reduce the power consumption of the medical sensor system 10, but may also reduce the SNR. In certain embodiments the first and the second non-sampling periods 146 and 148 may be between 500 and 2000 μ seconds, between 750 and 1500 μ seconds, or between 900 and 1100 μ seconds. Additionally, the duration of each of the first and the second non-sampling periods 146 and 148 may be between approximately 15 percent and 45 percent, 20 percent and 40 percent, or 30 percent and 35 percent of the duration of the multiplexing period 136. Accordingly, a longer duration of the first and the second non-sampling periods 146 and 148 increases the duration of the dark period 149. Thus, in embodiments in which the power-reducing timing cycle includes the first and the second non-sampling periods 146 and 148, the duration of each emitting period (e.g., the first red period 140 and the first IR period 144) may be between approximately five percent and 30 percent, 10 percent and 25 percent, or 15 percent and 20 percent of the duration of each dark period 149.

As noted above, FIG. 5 illustrates one embodiment of a method 180 for measuring one or more operating parameters of the medical sensor 14. The method 180 may include measuring the current of the emitter 20 at during each emitting period of a power-reducing timing cycle (e.g., the power-reducing timing cycle of FIG. 3) (block 182) and measuring the voltage of the emitter 20 just during each emitting period of the power-reducing timing cycle (block 184) to determine the LED fault conditions and the medical sensor 14 connect/disconnect status. The method 180 may also include measuring the current of the emitter 20 only during each dark period of the power-reducing timing cycle (block 256) to determine the emitter 20 and/or the LED drive 26 fault conditions. Furthermore, the method 180 may include measuring the voltage of the detector 22 and the voltage supply 70 during a dark period of the power-reducing timing cycle (block 186). For example, the voltage of the detector 22 and the voltage supply 70 may be measured during a dark period following a red emitting period or an IR emitting period.

As discussed above with respect to FIG. 3, the patient monitor 12 may compare the measurement of the operating parameter to an upper and/or a lower threshold, which may be specific for the operating parameter and/or specific for the particular medical sensor 14. Accordingly, following each measurement, the patient monitor 12 may determine whether the measured operating parameter is within a predetermined range (block 104). If the patient monitor 12 determines that the measurement of the operating parameter is within the predetermined range, the patient monitor 12 may continue the power-reducing timing cycle for the medical sensor 14 (block 106). However, if the operating parameter is not within the predetermined range, the method 90 may include resetting the medical sensor 14 (block 108) and determining whether the medical sensor 14 reset within a predetermined time (block 110). If the medical sensor 14 resets within the predetermined time, the patient monitor 12 may restart the multiplexing period 136, and thus, may begin by measuring the current of the emitter 20 at the start of each emitting period (block 182). However, in other embodiments, the patient monitor 12 may resume the multiplexing period 136 from the period when the medical sensor 14 was reset. If the patient monitor 12 determines that the medical sensor 14 failed to reset within the predetermined time, the patient monitor 12 may transmit an error signal to the DSP 66 (block 112).

One embodiment of the manner in which the operating parameters of the medical sensor 14 and/or the patient monitor 12 may be measured in accordance with the method of FIG. 5 is depicted as a timing diagram 200 in FIG. 6. As noted above, the operating parameters may include the current and/or voltage of the emitter 20 during the emitting periods, the current of the emitter 20 during the dark periods, the voltage of the detector 22, and/or the voltage of a voltage supply 72 provided to the medical sensor 14. Accordingly, the timing diagram 200 depicts current pulses 202 (e.g., the LED current signals 28) provided to the emitter 20 of the medical sensor 14 as a function of time 204 and the time points for measuring an operating parameter of the medical sensor 14. The timing diagram 200 also illustrates the multiplexing period 136, as described above with respect to FIG. 4, and thus, includes the first and the second dark periods 138 and 142, the first red period 140, the first IR period 144, and the first and the second non-sampling periods 146 and 148, which following the first red period 140 and the first IR period 144, respectively.

As discussed in detail above, the patient monitor 12 may measure one or more operating parameters of the medical sensor 14 throughout each multiplexing period 136 to determine whether the medical sensor 14 is properly connected and/or whether the components of the medical sensor 14 (e.g., the emitter 20 or the detector 22) and/or the voltage supply 72 of the patient monitor 12 are properly functioning. For example, the ADC 76 may measure the current of the red and IR LEDs of the emitter 20 (e.g., the LED current signals 28) during the respective emitting period. In particular, the current may be measured during a first red current interval 208 and a first IR current interval 210. In certain embodiments, the first red and IR current intervals 208 and 210 may occur shortly after the red and IR LED is turned on, respectively. For example, the first red and IR current intervals 208 and 210 may begin between approximately 0 μ seconds and 100 μ seconds, 20 μ seconds and 80 μ seconds , or 30 μ seconds and 70 μ seconds after the red and IR LED is turned on, respectively. Furthermore, the duration of the red and IR current intervals 208 and 210 may be selected for a desired number of current measurements. For example, the ADC 76 may digitize between approximately 2 and 30 measurements or 10 to 20 measurements, which may depend on the duration of the first red and IR current intervals 208 and 210. In certain embodiments, duration of the red and IR current intervals 208 and 210 may be between approximately 0 to 250 μ seconds, 0 to 100 μ seconds, or 0 to 75 μ seconds. Furthermore, the duration of the red and the IR current intervals 208 and 210 may be between approximately 1 percent and 5 percent of the duration of the multiplexing period 136.

Additionally, the patient monitor 12 may measure the current of the red and IR LEDs during the dark periods to determine LED fault conditions. In particular, the current of the red and IR LEDs of the emitter 20 may be measured during a second red current interval 212 and a second IR current interval 214, respectively. The second red and IR current intervals 212 and 214 may occur shortly after the red and IR LED is turned off, respectively. For example, the second red and IR current intervals 212 and 214 may occur between approximately 0 to 20 μ seconds, 1 to 10 μ seconds, or 2 to 5 μ seconds after the respective LED is turned off. In certain embodiments, the ADC 76 may digitize between approximately 1 and 10 measurements or 2 to 5 measurements. In one embodiment, the patient monitor 12 may only measure the current of the red LED just after (e.g., between approximately 1 μ second and 5 μ seconds) it has been turned off.

Additionally, the patient monitor 12 may measure the voltage of the red and IR LEDs during a red voltage interval 216 and an IR voltage interval 218, respectively, to determine whether the voltages are within a predetermined range. For example, the ADC 76 may measure the voltage of the red and IR LEDs during their respective emitting periods. In certain embodiments, the voltage may be measured shortly before the LED is turned off. For example, the red and IR voltage may be measured between approximately 0 to 30 μ seconds, 5 to 20 μ seconds, or 10 to 20 μ seconds before the red and IR LED is turned off. The patient monitor 12 may also measure the voltage of the detector 22 and the voltage supply 72 during the multiplexing period 136 to determine whether each are within a normal operating range. While any number of measurements are presently contemplated, in certain embodiments, the voltage of the detector 22 and the voltage supply 72 may be measured only once during each multiplexing period 136. For example, the ADC 76 may measure the voltage of both the detector 22 and the voltage supply 72 during an interval 220 that occurs shortly after the red or the IR LED is turned off. Furthermore, for embodiments in which the multiplexing cycle 136 is synchronized with pulse data from an external source, such as an ECG sensor, the patient monitor 12 may measure the signal from the external source at the beginning of each emitting period to determine that a valid signal is present. For example, the signal may be measured over intervals 222, which occur shortly after (e.g., between approximately 0 μ seconds and 100 μ seconds, 20 μ seconds and 80 μ seconds, or 30 μ seconds and 70 μ seconds) the red or IR LED is turned on.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A method, comprising:
driving an emitter of a sensor in accordance with a power-reducing timing cycle using drive circuitry;
wherein the power-reducing timing cycle comprises emitting periods in which the emitter emits light and dark periods in which the emitter does not emit light; and
wherein a duration of each dark period is longer than a duration of each emitting period;
transforming, using a current-to-voltage converter, a first photocurrent signal from a detector of the sensor during the emitting periods into a first output voltage signal; and
calculating, using a processor, a physiological parameter of a patient based at least in part upon the first output voltage signal.

2. The method of claim 1, comprising transforming, using the current-to-voltage converter, a second photocurrent signal from the detector during the dark periods into a second output voltage signal and calculating, using the processor, the physiological parameter of the patient based at least in part upon the first and the second output voltage signal.

3. The method of claim 2, comprising digitizing, using an analog-to-digital converter, at least two measurements of the first output voltage signal and at least two measurements of the second output voltage signal during each emitting period and each dark period, respectively.

4. The method of claim 1, wherein the duration of each emitting period is between approximately 10 percent and 20 percent of the duration of each dark period.

5. The method of claim 1, comprising measuring, using the processor, an operating parameter of the sensor during at least one emitting period or dark period of the power-reducing timing cycle.

6. The method of claim 5, wherein the operating parameter comprises a current of the emitter, a voltage of the emitter, or a voltage of the detector.

7. The method of claim 5, comprising determining, using the processor, whether the measurement of the operating parameter is within a predetermined threshold range.

8. The method of claim 7, comprising generating an error signal, using the processor, in response to determining that the measurement of the operating parameter is not within the predetermined threshold range.

9. The method of claim 7, comprising adjusting a driving current of the emitter, using the drive circuitry, in response to determining that the operating parameter is not within the threshold range.

10. A system, comprising:
a sensor comprising an emitter configured to emit one or more wavelengths of light and a detector configured to detect the one or more wavelengths of light to measure a physiological parameter of a patient;
a patient monitor operatively coupled to the sensor, wherein the patient monitor comprises:
driving circuitry configured to drive the emitter of the sensor in accordance with a power-reducing timing cycle, wherein the power-reducing timing cycle comprises emitting periods in which the emitter emits the one or more wavelengths of light and dark periods in which the emitter does not emit the one or more wavelengths of light, and wherein a duration of each dark period is longer than a duration of each emitting period;
a current-to-voltage converter configured to convert a first photocurrent signal from the detector during the emitting periods into an output voltage signal; and
a processor configured to calculate the physiological parameter of the patient based at least in part upon the output voltage signal.

11. The system of claim 10, wherein the duration of each emitting period is between approximately 10 percent and 20 percent of the duration of each dark period.

12. The system of claim 10, wherein the patient monitor comprises an analog-to-digital converter configured to digitize at least two measurements of the output voltage signal during each emitting period.

13. The system of claim 12, wherein the patient monitor comprises a first timer, wherein the first timer is programmed with timing information for controlling when the analog-to-digital converter digitizes the at least two measurements of the output voltage signal.

14. The system of claim 10, wherein the patient monitor comprises an analog-to-digital converter configured to sample and digitize a measurement of an operating parameter of the sensor during at least one period of the power-reducing timing cycle.

15. The system of claim 14, wherein the processor is configured to determine whether the measurement of the operating parameter is within a predetermined threshold range.

16. The system of claim 14, wherein the patient monitor comprises a display, and wherein the processor is configured to cause the display to display an error message in response to determining that the operating parameter of the sensor is outside of the threshold range.

17. A tangible, non-transitory, machine-readable medium comprising code executable by a processor to perform the acts of:
driving an emitter of a sensor in accordance with a power-reducing timing cycle, wherein the power-reducing timing cycle comprises emitting periods in which the emitter emits light and dark periods in which the emitter does not emit light, and wherein a duration of each dark period is longer than a duration of each emitting period;
transforming a photocurrent signal generated by a detector of the sensor during the emitting periods into an output voltage signal; and
calculating a physiological parameter of a patient based at least in part upon the output voltage signal.

18. The tangible, non-transitory, machine-readable medium of claim 17, comprising code executable by the processor to perform the acts of:
digitizing two or more measurements of the output voltage signal during each emitting period;
averaging the two or more measurements of the output voltage signal; and
calculating the physiological parameter of the patient based at least in part upon the average of the two or more measurements.

19. The tangible, non-transitory, machine-readable medium of claim 17, comprising code executable by the processor to perform the acts of:
measuring an operating parameter of the sensor during at least one period of the power-reducing timing cycle; and
determining whether the measurement of the operating parameter of the sensor is within a predetermined threshold range.

20. The tangible, non-transitory, machine-readable medium of claim 19, comprising code executable by the processor to perform the acts of:
generating an error signal, or adjusting a driving current provided to the emitter of the sensor, or a combination thereof, in response to determining that the measurement of the operating parameter of the sensor is outside of the predetermined threshold range.
